(19) United States
(12) Patent Application Publication
(10) Pub. No.: US 2023/0192680 A1
(43) Pub. Date: Jun. 22, 2023

(54) NEW AGENT FOR PROMOTING PHOSPHORYLATION OF eIF2a

(71) Applicant: ER Stress Research Institute, Inc., Tokushima (JP)

(72) Inventor: Seiichi OYADOMARI, Tokushima (JP)

(73) Assignee: ER Stress Research Institute, Inc., Tokushima (JP)

(21) Appl. No.: 17/435,592

(22) PCT Filed: Mar. 10, 2020
(86) PCT No.: PCT/JP2020/010375

§ 371 Date: Mar. 9, 2022

(30) Foreign Application Priority Data
Mar. 11, 2019 (JP) 2019-068706
Nov. 15, 2019 (JP) 2019-219142

(51) Int. Cl. C07D 471/04; C07D 401/04; C07D 401/14

(52) U.S. Cl. CPC C07D 471/04; C07D 401/04; C07D 401/14

(57) ABSTRACT

A compound represented by the general formula (1) or a pharmacologically acceptable salt thereof is a novel eIF2α phosphorylation promotor:

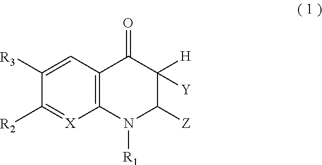

where: R₁ represents a substituted or unsubstituted aryl group, a substituted or unsubstituted aralkyl group, a substituted or unsubstituted cycloalkyl group, or a substituted or unsubstituted aromatic heterocyclic group; R₂ represents a substituted or unsubstituted aromatic heterocyclic group; R₃ represents a hydrogen atom, a hydroxy group, or a halogen atom; substituents of the aryl group, the aralkyl group, and the cycloalkyl group of R₁ are each independently a halogen atom, a hydroxy group, a lower alkyl group having 1 to 4 carbon atoms, or an aralkyl group; substituents of the aromatic heterocyclic groups of R₁ and R₂ are each independently a substituted or unsubstituted benzyl group, a hydroxy group, an alkyl group having 1 to 4 carbon atoms, a halogen atom, or an alkyloxy group having 1 to 4 carbon atoms; X represents a nitrogen atom or a methine group; and Y and Z both represent a hydrogen atom or are paired to represent a single bond.

### TECHNICAL FIELD

The present invention relates to a novel promotor of eIF2α phosphorylation. An eIF2α phosphorylation promotor of the present invention has a function of regulating an integrated stress response. Further, the eIF2α phosphorylation promotor of the present invention suppresses Th17 cell differentiation, and hence serves as a preventive or therapeutic agent for an autoimmune disease or a persistent inflammatory disease.

### BACKGROUND ART

A living body is constantly exposed to a wide variety of stresses from an outside world, and normally maintains homeostasis thereagainst through regulatory mechanisms, such as an endocrine system and a nervous system. When attention is focused on cells that make up the body, factors of the stresses include viral infection, nutritional deficiency (amino acids, sugar, or serum), oxygen deficiency, heat shock, oxidative stress, and the like.

Many findings have been published in recent years about how cells respond when exposed to those internal and external stresses. For example, there are known an endoplasmic reticulum stress response for dealing with an abnormal structure of a protein, and an integrated stress response involved in suppression of translation of eIF2α, for dealing with a wide range of internal and external stresses, such as viral infection, nutritional deficiency, and oxidative stress. As illustrated in FIG. 1, the integrated stress response and the endoplasmic reticulum stress response are stress response reactions having a PERK pathway in common. In addition, the endoplasmic reticulum stress response and the integrated stress response allow cells to adapt to their environment by regulating their function in response to a change in the environment.

With regard to the endoplasmic reticulum stress response, its activation mechanism is broadly divided into three routes. In these routes, an endoplasmic reticulum stress-transmitting protein (Irel, ATF6, or PERK), respectively, transmit information in the endoplasmic reticulum to the cytosol and the nucleus (see FIG. 2). For example, PERK activated by sensing endoplasmic reticulum stress phosphorylates eukaryotic initiation factor 2α subunit (eIF2α). As a result, the phosphorylated eIF2α causes global translational suppression to suppress protein synthesis, to thereby alleviate a load on the endoplasmic reticulum.

Meanwhile, the phosphorylated eIF2α induces expression of a transcription factor ATF4 (activating transcription factor 4) to activate amino acid metabolism and redox metabolism, which are needed for stress adaptation, and when repair is unlikely, induces expression of C/EBP homologous protein (CHOP), which is an apoptosis-related molecule, to cause cell death.

In the integrated stress response, as illustrated in FIG. 1, in addition to the above-mentioned PERK, three stress-transmitting proteins (eIF2α kinases), i.e., double-stranded RNA dependent protein kinase (PKR), heme-regulated inhibitor (HRI), and general control non-derepressible 2 (GCN2) function. As in the case of the endoplasmic reticulum stress response, those proteins sense internal and external stresses and modify eIF2α by phosphorylation to stop protein synthesis. Then, as in the case of the endoplasmic reticulum stress response, the phosphorylated eIF2α induces expression of the transcription factor ATF4 (activating transcription factor 4) to activate amino acid metabolism and redox metabolism, which are needed for stress adaptation, and when repair is unlikely, induces expression of C/EBP homologous protein (CHOP), which is an apoptosis-related molecule, to cause cell death. As described above, responses are made to various stress stimuli.

When the phosphorylation of eIF2α by the endoplasmic reticulum stress response and the integrated stress response is promoted, endoplasmic reticulum stress can be alleviated. It is reported that, as a result, cell death resulting from endoplasmic reticulum stress can be avoided, and the onset of various diseases (e.g., metabolic disease, cranial nerve disease, cancer, and immunological disease) can be suppressed (Patent Literature 1). Recently, drug discovery targeting the endoplasmic reticulum stress response and the integrated stress response has also been attracting attention (Patent Literature 2). For example, it has been found that administration of low-molecular-weight compounds called chemical chaperones to a mouse model of type 2 diabetes ameliorated its pathological conditions, such as insulin resistance (Non-Patent Literature 1). In addition, salubrinal, a low-molecular-weight compound, suppresses eIF2α dephosphorylation (consequently promoting eIF2α phosphorylation) to alleviate endoplasmic reticulum stress. It is reported that, as a result, nerve cell death resulting from endoplasmic reticulum stress can be avoided (Non-Patent Literature 2).

As described above, progress is being made in drug discovery research targeting the endoplasmic reticulum stress response and the integrated stress response. However, no disease therapeutic agent using an eIF2α phosphorylation promotor has yet been developed.

### CITATION LIST

### Patent Literature

Patent Literature 1: JP 2017-193527 A
Patent Literature 2: JP 2014-512008 A

### Non-Patent Literature

Non-Patent Literature 1: Ozcan et al. Science 313, 1173-1140 (2006)
Non-Patent Literature 2: Boyce M I et al, Science, 2005 Feb. 11; 307 (5711): 935-9. A selective inhibitor of eIF2alpha dephosphorylation protects cells from ER stress.

### SUMMARY OF INVENTION

### Technical Problem

An objective of the present invention is to provide a novel quinolone skeleton compound capable of promoting eIF2α phosphorylation to suppress endoplasmic reticulum stress, and further capable of suppressing differentiation into Th17 cells.

### Solution to Problem

The inventor of the present invention has found that a quinolone skeleton compound represented by the following general formula (1) is free of topoisomerase-inhibiting activity resulting in an antibacterial action, but has eIF2α phosphorylation-promoting activity, thereby suppressing endoplasmic reticulum stress, and further has Th17 cell differentiation-suppressing activity. Thus, the present invention has been completed.

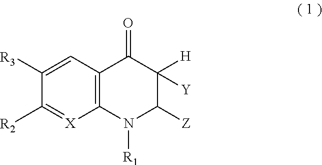

where: R₁ represents a substituted or unsubstituted aryl group, a substituted or unsubstituted aralkyl group, a substituted or unsubstituted cycloalkyl group, or a substituted or unsubstituted aromatic heterocyclic group; R₂ represents a substituted or unsubstituted aromatic heterocyclic group; R₃ represents a hydrogen atom, a hydroxy group, or a halogen atom; substituents of the aryl group, the aralkyl group, and the cycloalkyl group of R₁ are each independently a halogen atom, a hydroxy group, a lower alkyl group having 1 to 4 carbon atoms, or an aralkyl group; substituents of the aromatic heterocyclic groups of R₁ and R₂ are each independently a substituted or unsubstituted benzyl group, a hydroxy group, an alkyl group having 1 to 4 carbon atoms, a halogen atom, or an alkyloxy group having 1 to 4 carbon atoms; X represents a nitrogen atom or a methine group; and Y and Z both represent a hydrogen atom or are paired to represent a single bond.

That is, a gist of the present invention is as described below.

(1) A compound represented by the general formula (1) or a pharmacologically acceptable salt thereof:

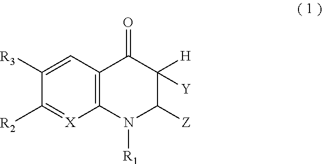

where: R₁ represents a substituted or unsubstituted aryl group, a substituted or unsubstituted aralkyl group, a substituted or unsubstituted cycloalkyl group, or a substituted or unsubstituted aromatic heterocyclic group; R₂ represents a substituted or unsubstituted aromatic heterocyclic group; R₃ represents a hydrogen atom, a hydroxy group, or a halogen atom; substituents of the aryl group, the aralkyl group, and the cycloalkyl group of R₁ are each independently a halogen atom, a hydroxy group, a lower alkyl group having 1 to 4 carbon atoms, or an aralkyl group; substituents of the aromatic heterocyclic groups of R₁ and R₂ are each independently a substituted or unsubstituted benzyl group, a hydroxy group, an alkyl group having 1 to 4 carbon atoms, a halogen atom, or an alkyloxy group having 1 to 4 carbon atoms; X represents a nitrogen atom or a methine group; and Y and Z both represent a hydrogen atom or are paired to represent a single bond.
(2) The compound or the pharmacologically acceptable salt thereof according to the above-mentioned item (1), wherein the substituted or unsubstituted aryl group is a 2-methylphenyl group, a p-hydroxyphenyl group, a p-fluorophenyl group, or a 2-chloro-5-methylphenyl group.
(3) The compound or the pharmacologically acceptable salt thereof according to the above-mentioned item (1) or (2), wherein the substituted or unsubstituted aralkyl group is a benzyl group or a phenyl-1,1-dimethylmethyl group.
(4) The compound or the pharmacologically acceptable salt thereof according to any one of the above-mentioned items (1) to (3), wherein the substituted or unsubstituted cycloalkyl group is a cyclohexyl group.
(5) The compound or the pharmacologically acceptable salt thereof according to any one of the above-mentioned items (1) to (4), wherein the substituted or unsubstituted aromatic heterocyclic groups are each a 4-pyridyl group, a 4-pyrazolyl group, a 3,5-dimethyl-4-pyrazolyl group, or a 1-N-benzyl-4-pyrazolyl group.
(6) The compound or the pharmacologically acceptable salt thereof according to any one of the above-mentioned items (1) to (5), wherein the X represents a nitrogen atom.
(7) The compound or the pharmacologically acceptable salt thereof according to any one of the above-mentioned items (1) to (5), wherein the X represents a methine group.
(8) The compound or the pharmacologically acceptable salt thereof according to any one of the above-mentioned items (1) to (7), wherein the Y and Z are paired to represent a single bond.
(9) The compound or the pharmacologically acceptable salt thereof according to the above-mentioned item (1), wherein R₁ represents a substituted or unsubstituted aryl group, R₂ represents an unsubstituted aromatic heterocyclic group, R₃ represents a hydrogen atom, X represents a nitrogen atom, and Y and Z are paired to represent a single bond.
(10) The compound or the pharmacologically acceptable salt thereof according to the above-mentioned item (1), wherein R₁ represents a substituted or unsubstituted phenyl group, R₂ represents an unsubstituted pyridyl group, R₃ represents a hydrogen atom, X represents a nitrogen atom, and Y and Z are paired to represent a single bond.
(11) The compound or the pharmacologically acceptable salt thereof according to the above-mentioned item (1), wherein R₁ represents a phenyl group having a substituent, R₂ represents an unsubstituted pyridyl group, R₃ represents a hydrogen atom, X represents a nitrogen atom, and Y and Z are paired to represent a single bond.
(12) The compound or the pharmacologically acceptable salt thereof according to the above-mentioned item (1), wherein R₁ represents a phenyl group having as substituents an alkyl group having 1 to 4 carbon atoms and a halogen atom, R₂ represents an unsubstituted pyridyl group, R₃ represents a hydrogen atom, X represents a nitrogen atom, and Y and Z are paired to represent a single bond.
(13) An eIF2α phosphorylation promotor, including as an active ingredient the compound or the pharmacologically acceptable salt thereof of any one of the above-mentioned items (1) to (12).
(14) The eIF2α phosphorylation promotor according to the above-mentioned item (13), wherein the eIF2α phosphorylation promotor is free of topoisomerase-inhibiting activity.
(15) The eIF2α phosphorylation promotor according to the above-mentioned item (13) or (14), wherein the eIF2α phosphorylation promotor further has Th17 cell differentiation-suppressing activity.
(16) A preventive or therapeutic agent for an autoimmune disease or a persistent inflammatory disease, including as an active ingredient the eIF2α phosphorylation promotor of any one of the above-mentioned items (13) to (15).

### Advantageous Effects of Invention

The quinolone skeleton compound represented by the general formula (1), which is the compound of the present invention, promotes eIF2α phosphorylation to suppress endoplasmic reticulum stress, and also suppresses differentiation into Th17 cells (helper T17 cells).

Cytokines produced by helper T cells form a cascade and are involved in a wide range of inflammations. In particular, Th17 cells are known to produce a large number of cytokines, including IL-17 and TNF-α, to thereby induce an autoimmune disease, such as rheumatoid arthritis, multiple sclerosis, Crohn's disease, ulcerative colitis, or psoriasis (Jpn. J. Clin. Immunol., 33(5) 262-271). Accordingly, the compound of the present invention, which suppresses the induction of differentiation into Th17 cells, can be used as a preventive or therapeutic agent for such autoimmune disease. In addition, the compound of the present invention suppresses the induction of differentiation into Th17 cells like halofuginone, and hence, like halofuginone, can be used as a preventive or therapeutic agent for a persistent inflammatory disease as well as the autoimmune disease.

In addition, it is known that disruption of the endoplasmic reticulum stress response or the integrated stress response induces diseases such as diabetes, arteriosclerosis, a neurodegenerative disease (e.g., Parkinson's disease, polyglutamine disease, Alzheimer's disease, or amyotrophic lateral sclerosis), cancer, and an autoimmune disease (Oyadomari et al, Cell Metab 7, 520-532 (2008); Baird et al, Adv Nutr 3, 307-321 (2012); Costa-Mattioli et al. Nature 436, 1166-1173 (2005); Sidrauski et al. Elife 2, e00498 (2013); Dey et al. J Clin Invest, 125, 2592-2608 (2015); Munn et al. Immunity, 22, 633-642 (2005); Oyadomari et al, FAEB J. 30, 798-812 (2016); Biomedical Gerontology 37(3); 9-16, 2013). The compound of the present invention normalizes a disrupted endoplasmic reticulum stress response by promoting eIF2α phosphorylation, and hence can be used as a preventive or therapeutic agent for those diseases.

In addition, some compounds each having a quinolone skeleton show antibacterial activity based on a topoisomerase inhibitory action, but the compound of the present invention does not show topoisomerase-inhibiting activity. Accordingly, the compound of the present invention cannot be used as an antibacterial agent or anticancer agent based on topoisomerase-inhibiting activity.

### BRIEF DESCRIPTION OF DRAWINGS

FIG. 1 is an illustration of the flow of intracellular communication by a kinase to be activated by endoplasmic reticulum stress (PERK/PEK), a kinase to be activated by amino acid depletion (GCN2), a kinase to be activated by Fe³⁺ deficiency (HRI), and a kinase to be activated by viral infection (PKR).

FIG. 2 is an illustration of the mechanism of protein production control by endoplasmic reticulum stress.

FIG. 3 shows the eIF2α phosphorylation-promoting effect of a compound (No. 1) of the present invention.

FIG. 4 shows that the compound (No. 1) of the present invention is free of topoisomerase-inhibiting activity.

FIG. 5 shows that the compound (No. 1) of the present invention has Th17 cell differentiation-suppressing activity.

### DESCRIPTION OF EMBODIMENTS

In the general formula (1) representing a compound of the present invention, an aryl group represented by R₁ represents a phenyl group or a naphthyl group, and may be substituted with one or more substituents. Examples of the substituents may include: a hydroxy group; alkyl groups each having 1 to 4 carbon atoms, such as a methyl group, an ethyl group, and a propyl group; halogen atoms, such as a fluorine atom, a chlorine atom, and a bromine atom; and alkyloxy groups each having 1 to 4 carbon atoms, such as a methoxy group and an ethoxy group. Preferred examples of the substituted or unsubstituted aryl group represented by R₁ may include an o-methylphenyl group, a p-hydroxyphenyl group, a p-fluorophenyl group, and a 2-chloro-5-methylphenyl group.

An aralkyl group represented by R₁ may be preferably, for example, a phenylalkyl group, such as a phenylmethyl group, a phenylethyl group, or a phenylpropyl group. Examples of a substituent of the phenyl group and a substituent of the alkyl group include the above-mentioned substituents. Preferred examples of the substituted or unsubstituted aralkyl group represented by R₁ may include a benzyl group and a phenyl-1,1-dimethylmethyl group.

A cycloalkyl group represented by R₁ may be preferably, for example, a saturated or unsaturated alicyclic group having 5 to 7 carbon atoms, such as a cyclohexyl group or a cyclopentyl group. Examples of a substituent thereof include the above-mentioned substituents.

An aromatic heterocyclic group represented by R₁ may be preferably, for example, a nitrogen-containing five- or six-membered ring group, such as a pyrazolyl group, an imidazole group, a pyrrole group, a pyridyl group, or a pyrimidyl group. Examples of a substituent thereof may include: a substituted or unsubstituted benzyl group; a hydroxy group; alkyl groups each having 1 to 4 carbon atoms, such as a methyl group, an ethyl group, and a propyl group; halogen atoms, such as a fluorine atom, a chlorine atom, and a bromine atom; and alkyloxy groups each having 1 to 4 carbon atoms, such as a methoxy group and an ethoxy group. A preferred example of the substituted or unsubstituted aromatic heterocyclic group represented by R₁ may be a 1-N-benzyl-pyrazolyl group.

An aromatic heterocyclic group represented by R₂ may be, for example, a nitrogen-containing five- or six-membered ring group, such as a pyrazolyl group, an imidazolyl group, a pyrrole group, a pyridyl group, or a pyrimidyl group. Examples of a substituent thereof may include: a substituted or unsubstituted benzyl group; a hydroxy group; alkyl groups each having 1 to 4 carbon atoms, such as a methyl group, an ethyl group, and a propyl group; halogen atoms, such as a fluorine atom, a chlorine atom, and a bromine atom; and alkyloxy groups each having 1 to 4 carbon atoms, such as a methoxy group and an ethoxy group. Preferred examples of the substituted or unsubstituted aromatic heterocyclic group represented by R₂ may include a 4-pyridyl group, a 4-pyrazole group, and a 3,5-dimethyl-4-pyrazole group.

A halogen atom represented by R₃ may be, for example, a fluorine atom, a chlorine atom, or a bromine atom. A preferred example of R₃ may be a hydrogen atom or a fluorine atom.

X represents a nitrogen atom or a methine group, and a preferred example thereof may be a methine group.

Y and Z both represent a hydrogen atom or are paired to represent a single bond, and may be preferably, for example, paired to represent a single bond.

Of the compounds each represented by the general formula (1), a particularly preferred compound is a compound in which R₁ represents a substituted or unsubstituted aryl group, in particular, a substituted or unsubstituted phenyl group, in particular, a phenyl group having a substituent, in particular, a phenyl group having as substituents an alkyl group having 1 to 4 carbon atoms and a halogen atom, in particular, a phenyl group having as substituents a methyl group and a chlorine atom, R₂ represents an unsubstituted aromatic heterocyclic group, in particular, an unsubstituted pyridyl group, R₃ represents a hydrogen atom, X represents a nitrogen atom, and Y and Z are paired to represent a single bond.

The quinolone derivative that is the compound of the present invention may be synthesized using known general-purpose means (Molecules, 2016, 21, 268; doi: 10.3390/molecules21040268 or Synlett, 2012, 3, 448). For example, Compound No. 4 of the present invention may be synthesized in the following manner.

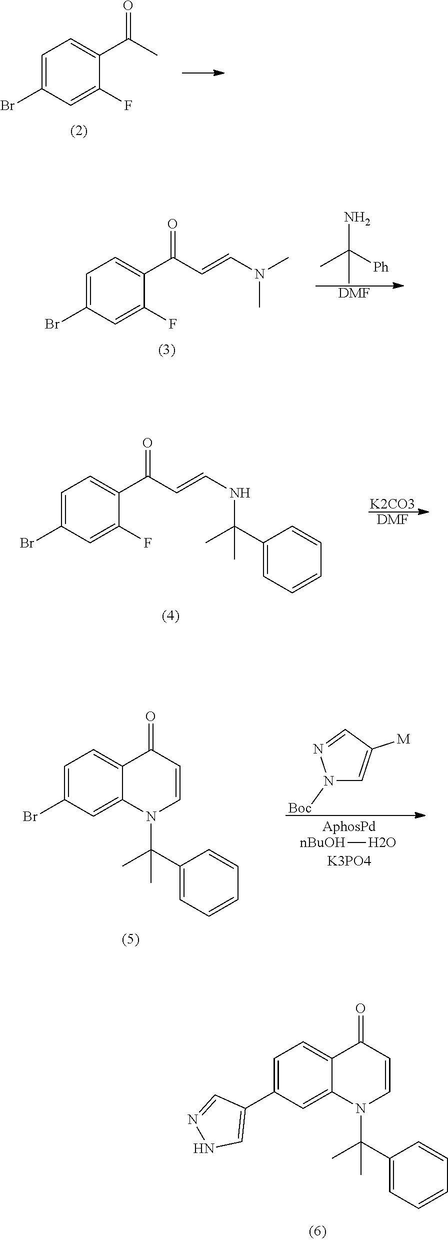

The term “eIF2α phosphorylation promotor” as used in the present invention refers to a compound that promotes eIF2α phosphorylation by activating an eIF2α kinase or suppressing eIF2α dephosphorylation.

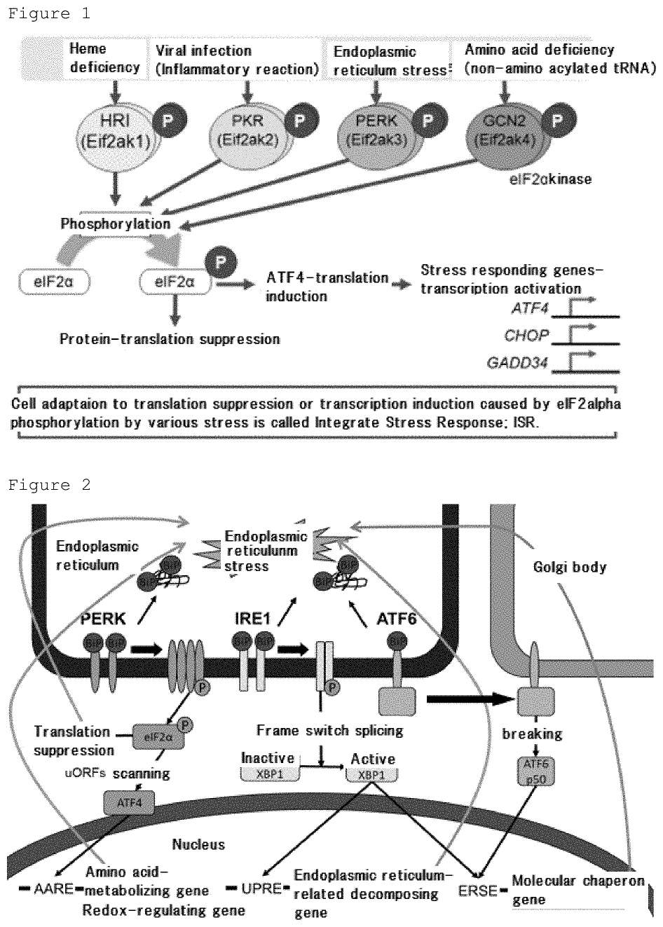

For example, as illustrated in FIG. 1, a kinase to be activated by endoplasmic reticulum stress (PERK/PEK), a kinase to be activated by amino acid depletion (GCN2), a kinase to be activated by Fe³⁺ deficiency (HRI), a kinase to be activated by viral infection (PKR), and the like acquire extremely strong abilities to phosphorylate eIF2α through dimerization and self-phosphorylation. Those activated kinases perform eIF2α phosphorylation. Further, after a stress response by eIF2α phosphorylation, the phosphorylated eIF2α is dephosphorylated by an eIF2α phosphatase (PP1: serine/threonine protein phosphatase type 1). Thus, through the actions of the eIF2α kinases and the phosphatase, eIF2α is phosphorylated to promote a stress response at the time of the above-mentioned stress stimuli.

The eIF2α phosphorylation inhibits the action of eIF2B, which is essential for starting translation, to thereby suppress translation. Thus, protein syntheses from many genes are stopped. When protein synthesis is stopped, endoplasmic reticulum stress is suppressed.

Meanwhile, the transcription factor ATF4 is regulated by the selection of an open reeding frame (ORF) encoding a gene downstream thereof, and quite opposite to other genes, translation thereof is induced, with the result that endoplasmic reticulum stress is enhanced to activate genes associated with metabolism, oxidative stress, and cell death.

As shown in FIG. 3, the compound of the present invention promotes eIF2α phosphorylation, and hence can be used as an endoplasmic reticulum stress suppressor like thapsigargin and halofuginone.

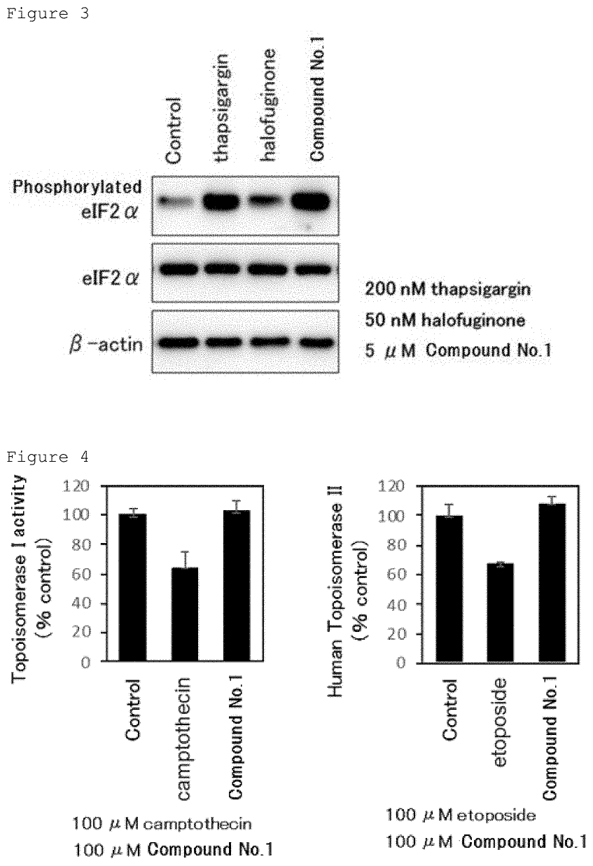

The term “topoisomerase-inhibiting activity” as used in the present invention refers to inhibiting a topoisomerase enzyme. The term “topoisomerase enzyme” collectively refers to enzymes each of which cleaves and religates one or both of double-stranded DNA, and topoisomerases are broadly classified into two types (type I and type II). A type I topoisomerase (topo I) in eukaryotic cells is inhibited by an anticancer agent irinotecan or topotecan. The type I topoisomerase is also inhibited by camptothecin having a similar structure. A type II topoisomerase (topo II) in eukaryotes is inhibited by an anticancer agent etoposide or teniposide. In addition, many bacteria have another kind of type II topoisomerase (DNA gyrase), which is inhibited by an antibiotic having a quinolone skeleton. However, contrary to conventional knowledge, as shown in FIG. 4, the quinolone skeleton compound of the present invention shows no inhibitory activity on the type I and type II topoisomerases while having a quinolone skeleton. That is, it is conceived that the compound of the present invention shows no antibacterial activity or anticancer activity based on a topoisomerase inhibitory action.

The term “Th17 cell differentiation-suppressing activity” as used in the present invention refers to an action of suppressing the induction of differentiation from naive T cells into Th17 cells. The Th17 cells are a subset of helper T cells (Th cells), which are a kind of white blood cells, and the Th17 cells have been newly discovered in recent years. The Th17 cells have an ability to produce interleukin (IL)-17, which is a cytokine, and are so called because of the ability. It is known that the Th17 cells produce inflammatory cytokines, such as IL-17, IL-21, IL-22, and TNF-α, to induce inflammation. There is a report that the Th17 cells are involved in an autoimmune disease, such as inflammatory bowel disease (e.g., Crohn's disease) or rheumatoid arthritis, or allergy. That is, an autoimmune disease, allergy, or a persistent inflammatory disease can be ameliorated or treated by suppressing differentiation into the Th17 cells.

As shown in FIG. 5, the compound of the present invention was revealed to have suppressive activity on differentiation into the Th17 cells. Accordingly, the compound of the present invention can prevent, ameliorate, or treat an autoimmune disease, allergy, or a persistent inflammatory disease like halofuginone.

The term “autoimmune disease” as used in the present invention refers to a disease in which an endogenous tissue is recognized as a foreign body to produce an antibody called an autoantibody or an immune cell, which targets and attacks a specific cell or tissue to cause inflammation and tissue damage. Examples thereof may include: type 1 diabetes; Basedow's disease; rheumatoid arthritis; inflammatory bowel disease, such as Crohn's disease or ulcerative colitis; multiple sclerosis; and psoriasis.

The term “persistent inflammatory disease” as used in the present invention refers to a new category of disease also known as “autoinflammatory disease”. The term refers to, for example, a disease in the case where persistent inflammation such as persistent fever or cyclic fever of unknown cause is present. The above-mentioned autoimmune disease is known as an abnormality in acquired immunity. In recent years, there has been revealed an “autoinflammatory disease” in which, owing to an abnormality in natural immunity, an inflammatory reaction occurs spontaneously, leading to organ damage. Its difference from the above-mentioned “autoimmune disease” is that, in the autoimmune disease, an autoantibody, autoreactive T lymphocytes, or the like is detected, making it easy to diagnose the autoimmune disease, whereas the autoinflammatory disease has a feature in that the autoantibody or the autoreactive T lymphocytes are not detected. Accordingly, clinical symptoms and a genetic test are important for the diagnosis of the autoinflammatory disease. The autoinflammatory disease encompasses, for example, the following diseases based on pathological classification according to Keio hospital information & patient assistance service (KOMPAS): As abnormal pathology having an influence on an inflammasome, there are given familial Mediterranean fever, hyper IgD syndrome, Muckle-Wells syndrome, familial cold autoinflammatory syndrome, chronic infantile neurological cutaneous articular syndrome (CINCA syndrome)/neonatal onset multisystem inflammatory disease (NOMID), NLRC4-MAS PLAID (PLCγ2 associated antibody deficiency and immune dysregulation), and APLAID (autoinflammation and PLCγ2 associated antibody deficiency and immune dysregulation). As pathology not involved in the inflammasome, there are given TRAPS (TNF receptor-associated periodic syndrome), pyogenic sterile arthritis, pyoderma gangrenosum, acne (PAPA syndrome), Blau syndrome, ADAM17 deletion, chronic recurrent multifocal osteomyelitis and congenital dyserythropoietic anemia (Majeed syndrome), DIRA (deficiency of the interleukin 1 receptor antagonist), DITRA (deficiency of IL-36 receptor antagonist), SLC29A3 mutation, CAMPS (CARD14 mediated psoriasis), cherubism, CANDLE (chronic atypical neutrophilic dermatotis with lipodystrophy), and COPA (coatomer protein complex, subunit alpha) deficiency. Further, it is considered that type 2 diabetes and arteriosclerosis are also encompassed in the autoinflammatory disease in a broad sense.

Further, the compound of the present invention normalizes the disruption of the endoplasmic reticulum stress response or the integrated stress response, and hence can also be used as a therapeutic agent for neurodegenerative diseases, such as Parkinson's disease, polyglutamine disease, Alzheimer's disease, and amyotrophic lateral sclerosis, various cancers, diabetes, and arteriosclerosis.

The present invention provides: a use of the compound represented by the general formula (1) for producing an eIF2α phosphorylation promotor; the compound represented by the general formula (1) for use in a method of promoting eIF2α phosphorylation; and a method of promoting eIF2α phosphorylation including administering an effective amount of the compound represented by the general formula (1) to a human having a disrupted or reduced endoplasmic reticulum stress response or integrated stress response.

The present invention also provides: a use of the compound represented by the general formula (1) for producing a preventive or therapeutic agent for an autoimmune disease, a persistent inflammatory disease, a neurodegenerative disease, cancer, diabetes, or arteriosclerosis; the compound represented by the general formula (1) for use in a method of preventing or treating an autoimmune disease, a persistent inflammatory disease, a neurodegenerative disease, cancer, diabetes, or arteriosclerosis; and a method of preventing or treating an autoimmune disease, a persistent inflammatory disease, a neurodegenerative disease, cancer, diabetes, or arteriosclerosis, the method including administering the compound represented by the general formula (1) in an amount effective for preventing or treating the autoimmune disease, the persistent inflammatory disease, the neurodegenerative disease, cancer, diabetes, or arteriosclerosis to a patient with the autoimmune disease, the persistent inflammatory disease, the neurodegenerative disease, cancer, diabetes, or arteriosclerosis.

The term “preventive or therapeutic agent” as used in the present invention refers to a drug to be used for the prevention or treatment of an autoimmune disease, an autoinflammatory disease, a neurodegenerative disease, cancer, diabetes, arteriosclerosis, or the like. Specific examples of preparations include: an oral preparation, such as a tablet, a capsule, a granule, a powder, or a syrup; an injection, such as intravenous injection, or intramuscular injection; and an intravenous drip infusion. Those preparations are each prepared using a generally used pharmaceutical carrier.

As the pharmaceutical carrier, there is used a substance that is commonly used in the field of pharmaceuticals and does not react with the compound of the present invention. Specific examples of the pharmaceutical carrier to be used in the production of the tablet, the capsule, the granule, or the powder include: excipients, such as lactose, corn starch, sucrose, mannitol, calcium sulfate, and crystalline cellulose; disintegrants, such as carmellose sodium, modified starch, and carmellose calcium; binders, such as methyl cellulose, gelatin, gum arabic, ethyl cellulose, hydroxypropyl cellulose, and polyvinylpyrrolidone; and lubricants, such as light anhydrous silicic acid, magnesium stearate, talc, and hydrogenated oil. The tablet may be coated with a normal coating agent by a well-known method.

Specific examples of the carrier to be used in the production of the syrup include: sweetening agents, such as sucrose, glucose, and fructose; suspending agents, such as gum arabic, tragacanth, carmellose sodium, methyl cellulose, sodium alginate, crystalline cellulose, and VEEGUM; and dispersants, such as a sorbitan fatty acid ester, sodium lauryl sulfate, and polysorbate 80.

The injection is generally prepared by dissolving the above-mentioned active ingredient in distilled water for injection, and as required, a solubilizing agent, a buffer, a pH adjuster, a tonicity agent, an analgesic, a preservative, or the like may be added. Further, the form of an injectable suspension obtained by suspending the compound in distilled water for injection or a plant oil may be adopted, and as required, a base, a suspending agent, a viscosity modifier, or the like may be added.

The dose of the compound of the present invention varies depending on the kind or severity of disease or symptoms, a dosage form, an administration route, and the like, and may be appropriately decided by a person skilled in the art. The dose may be, for example, 0.00001 mg or more, 0.0001 mg or more, 0.001 mg or more, 0.01 mg or more, 0.1 mg or more, or 1 mg or more as a daily dose, and may be, for example, 100 mg or less, 10 mg or less, 1 mg or less, 0.1 mg or less, 0.01 mg or less, 0.001 mg or less, or 0.0001 mg or less as a daily dose.

### EXAMPLES

The present invention is specifically described below by way of Examples and Test Examples. However, the present invention is not limited to these Examples and Test Examples.

### (Example 1) Synthesis of Compound (No. 1) of the Present Invention

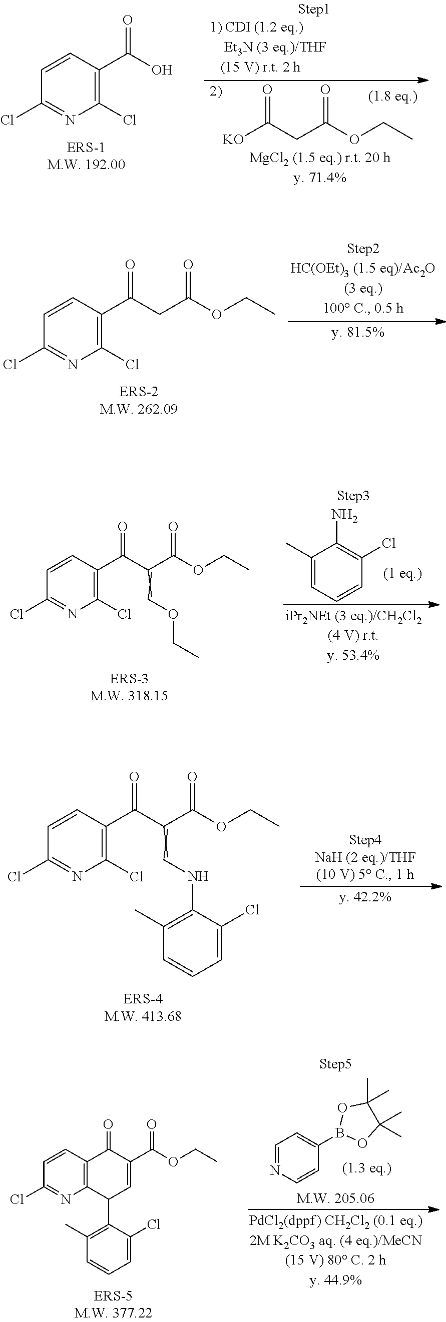

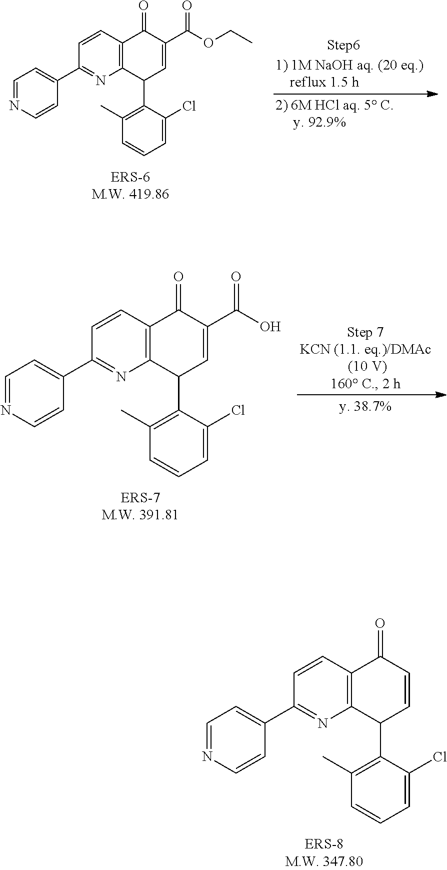 [SMILES: CCOC(=O)C1=CC(c2c(C)cccc2Cl)c2nc(-c3ccncc3)ccc2C1=O ; Cc1cccc(Cl)c1C1C=C(C(=O)O)C(=O)c2ccc(-c3ccncc3)nc21 ; Cc1cccc(Cl)c1C1C=CC(=O)c2ccc(-c3ccncc3)nc21]

### 1-(2-chloro-5-methylphenyl)-4-oxo-7-(4-pyridyl)-naphthyridine

(Compound No. 1 of the present invention) (compound ERS-8 in the stepwise scheme) was synthesized through seven steps shown in the stepwise scheme using commercially available 2,6-dichloronicotinic acid (compound ERS-1 in the stepwise scheme) as a raw material.

Step 1 is a carbon-increasing reaction using a malonic acid derivative, Step 2 is the synthesis of an ethoxyacrylic acid derivative using ethyl orthoformate, Step 3 is a substitution reaction with an aniline derivative, Step 4 is an intramolecular ring closure reaction for forming a 1,8-naphthyridine skeleton, Step 5 is a Suzuki-Miyaura cross-coupling reaction with a pyridine derivative, Step 6 is a hydrolysis reaction of an ester, and Step 7 is a decarboxylation reaction of a carboxylic acid. The target compound was purified by recrystallization from ethanol-water.

Step 1:

A 100 mL recovery flask was purged with nitrogen, and 2,6-dichloronicotinic acid (3.84 g, 20.0 mmol) was placed therein and dissolved by adding tetrahydrofuran (super dehydrated, 48.0 mL, 15 V). 1,1′-carbonyldiimidazole (3.28 g, 20.2 mmol) and triethylamine (7.1 mL, 50.1 mmol) were added at room temperature, and the mixture was stirred as it was for 2 hours. Monoethyl potassium malonate (5.18 g, 30.4 mmol) and anhydrous magnesium chloride (2.38 g, 25.0 mmol) were added to the reaction liquid, and the mixture was stirred at room temperature for 20 hours. The reaction liquid was poured into a 1 M aqueous solution of citric acid (100 mL), and extracted with ethyl acetate (120 mL). The organic layer was washed with saturated saline, and then dried over anhydrous magnesium sulfate. After separation by filtration, the solvent was concentrated under reduced pressure. The crude product was purified by being applied to a medium-pressure purification apparatus (silica gel: Kanto Chemical Co., Inc., neutral, spherical, 60N, 110 g). Elution was performed by a system of hexane:ethyl acetate (from 80:20 to 50:50), and the active fraction was concentrated under reduced pressure. The residue was dried under reduced pressure for 2 hours to produce 3.12 g of an ester ERS-2 as a colorless and transparent oily substance in a yield of 71.4%.

Data: ¹HNMR (CDCl3, δ in ppm): 1.35 (t, d=7.6 Hz, —CH2CH3-, 3H), 4.09 (s, —CH2-, 2H), 4.28 (q, d=7.6 Hz, —CH2CH3, 2H), 7.40 (d, J=8.0 Hz, ArH, 1H), 7.98 (d, J=8.0 Hz, ArH, 1H)

Step 2:

A 100 mL recovery flask was purged with nitrogen, and the ester ERS-2 (3.11 g, 11.9 mmol) was placed therein. Triethyl orthoformate (2.96 mL, 17.8 mmol) and acetic anhydride (3.37 mL, 35.7 mmol) were added at room temperature, and the mixture was heated and stirred in an oil bath at a temperature of 140° C. for 0.5 hour. The reaction liquid was concentrated under reduced pressure, and azeotroped with toluene three times to remove the reactants to the extent possible. The resultant crude product was purified by being applied to a medium-pressure purification apparatus (silica gel: Kanto Chemical Co., Inc., neutral, spherical, 60N, 100 g). Elution was performed by a system of hexane:ethyl acetate (from 80:20 to 50:50), and the active fraction was concentrated under reduced pressure. The residue was dried under reduced pressure for 2 hours to produce 3.08 g of an ethoxyacrylic acid derivative ERS-3 as a red-brown oily substance in a yield of 81.5%.

Data: ¹HNMR (CDCl3, δ in ppm): 1.17 (t, d=7.6 Hz, —CH2CH3-, 3H), 1.35 (t, d=7.6 Hz, —CH2CH3-, 3H), 4.13 (q, d=7.6 Hz, —CH2CH3, 2H), 4.35 (q, d=7.6 Hz, —CH2CH3, 2H), 7.33 (d, J=8.0 Hz, ArH, 1H), 7.98 (d, J=8.0 Hz, ArH, 1H), 7.87 (s, ═CH—, 1H)

Step 3:

A 100 mL recovery flask was purged with nitrogen, and the ethoxyacrylic acid derivative (3.07 g, 9.65 mmol) was placed therein and dissolved by adding dichloromethane (12.3 mL, 4 V). 2-chloro-6-methylaniline (1.17 mL, 9.65 mmol) and N,N-diisopropylethylamine (5.04 mL, 29.0 mmol) were added at room temperature, and the mixture was stirred as it was for 2 hours. The reaction liquid was concentrated under reduced pressure, and the resultant crude product was purified by being applied to a medium-pressure purification apparatus (silica gel: Kanto Chemical Co., Inc., neutral, spherical, 60N, 110 g). Elution was performed by a system of hexane:ethyl acetate (from 95:5 to 70:30), and the active fraction was concentrated under reduced pressure. The residue was dried under reduced pressure for 2 hours to produce 2.13 g of a substitution product ERS-4 as white powder in a yield of 53.4%.

Data: ¹HNMR (CDCl3, δ in ppm): 1.05 (t, d=7.2 Hz, —CH2CH3-, 3H), 2.46 (s, ArCH3, 3H), 4.03 (q, d=7.2 Hz, —CH2CH3, 2H), 7.11-7.22 (m, Ar2H, 2H), 7.28-7.38 (m, ArH and ═CH, 2H), 7.61 (d, j=8.4 Hz, ArH, 1H), 8.41 (d, J=8.4 Hz, ArH, 1H), 12.26 (d, d=14 Hz, NH, 1H)

Step 4:

A 100 mL recovery flask was purged with nitrogen, and the substitution product ERS-4 (2.12 g, 5.12 mmol) was placed therein and dissolved by adding tetrahydrofuran (21.2 mL, 10 V). The solution was cooled to 5° C. Sodium hydride (60% oil dispersion) (0.410 g, 10.2 mmol) was added, and the mixture was stirred as it was for 1 hour. Ice water (20 mL) was added to the reaction liquid, and then ethyl acetate (50 mL) was added for extraction. The separated organic layer was washed with saturated saline, and then dried over anhydrous magnesium sulfate. After separation by filtration, the solvent was evaporated under reduced pressure. The resultant crude product was purified by being applied to a medium-pressure purification apparatus (silica gel: Kanto Chemical Co., Inc., neutral, spherical, 60N, 45 g). Elution was performed by a system of hexane:ethyl acetate (from 80:20 to 50:50), and the active fraction was concentrated under reduced pressure. Diisopropyl ether (10 mL) was added to the resultant solid, and the solid was taken by filtration and dried under reduced pressure at 50° C. for 2 hours. 0.81 g of a ring closure product ERS-5 was obtained as pale yellow powder in a yield of 42.2%.

Data: ¹HNMR (DMSO-d6, δ in ppm): 1.27 (t, d=7.2 Hz, —CH2CH3-, 3H), 2.09 (s, ArCH3, 3H), 4.23 (q, d=7.2 Hz, —CH2CH3, 2H), 7.50-7.61 (m, Ar3H, 3H), 7.65 (d, j=8.8 Hz, ArH, 1H), 8.64 (d, J=8.8 Hz, ArH, 1H), 8.65 (s, ArH, 1H)

Step 5:

A 100 mL recovery flask was purged with nitrogen, and the ring closure product ERS-5 (0.800 g, 2.12 mmol) and 4-(4,4,5,5-tetramethyl-1,3,2-dioxaborolan-2-yl)pyridine (0.435 g, 2.76 mmol) were placed therein and dissolved by adding acetonitrile (12 mL, 15 V). A 2 M aqueous solution of potassium carbonate (4.24 mL, 8.48 mmol) was added, and then the solution was degassed by passing nitrogen therethrough for 3 minutes. Under a nitrogen atmosphere, PdCl₂ (dppf) CH₂Cl₂ (0.173 g, 0.212 mmol) was added, and the mixture was heated and stirred in an oil bath at a temperature of 80° C. for 2 hours. The reaction liquid was cooled to room temperature, ethyl acetate (50 mL), water (10 mL), and activated carbon were added, and the mixture was stirred for 20 minutes, followed by separation by filtration through Celite. The filtrate was subjected to liquid separation, and the organic layer was washed with saturated saline and then dried over anhydrous magnesium sulfate. After separation by filtration, the solvent was evaporated under reduced pressure. The resultant crude product was purified by being applied to a medium-pressure purification apparatus (silica gel: Kanto Chemical Co., Inc., neutral, spherical, 60N, 45 g). Elution was performed by a system of dichloromethane:methanol (from 99:1 to 90:10), and the active fraction was concentrated under reduced pressure. Diisopropyl ether (8 mL) was added to the resultant solid, and the solid was taken by filtration, washed with diisopropyl ether (2 mL), and then dried under reduced pressure at 50° C. for 2 hours. 0.40 g of a coupling product ERS-6 was obtained as pale yellow powder in a yield of 44.9%.

Data: ¹HNMR (DMSO-d6, δ in ppm): 1.29 (t, d=7.2 Hz, —CH2CH3-, 3H), 2.12 (s, ArCH3, 3H), 4.25 (q, d=7.2 Hz, —CH2CH3, 2H), 7.55-7.65 (m, Ar3H, 3H), 7.75 (2H, dd, J=4.4 Hz and 1.2 Hz, Py2H, 2H), 8.31 (d, j=8.0 Hz, ArH, 1H), 8.67 (dd, J=4.4 Hz and 1.2 Hz, Py2H, 2H), 8.74 (s, ArH, 1H), 8.77 (d, J=8.0 Hz, Ar1H, 1H)

Step 6:

The coupling product ERS-6 (0.38 g, 0.905 mmol) was placed in a 100 mL recovery flask, a 1 M aqueous solution of sodium hydroxide (18.1 mL, 18.1 mmol) was added, and the mixture was heated and stirred in an oil bath at a temperature of 120° C. for 1.5 hours. The reaction liquid was cooled to 5° C., 6 M hydrochloric acid was added to adjust the pH to from about 8 to about 9, and then extraction was performed with chloroform:methanol (10:1) three times. The organic layer was dried over anhydrous magnesium sulfate. After separation by filtration, the solvent was evaporated under reduced pressure. Diisopropyl ether (10 mL) was added to the resultant solid, and the solid was taken by filtration, washed with diisopropyl ether (3 mL), and then dried under reduced pressure at 50° C. for 2 hours. 0.33 g of a carboxylic acid ERS-7 was obtained as pale brown powder in a yield of 92.9%.

Data: ¹HNMR (DMSO-d6, δ in ppm): 2.11 (s, ArCH3, 3H), 7.55-7.66 (m, Ar3H, 3H), 7.79 (2H, dd, J=6.0 Hz and 1.2 Hz, Py2H, 2H), 8.47 (d, j=8.0 Hz, ArH, 1H), 8.71 (dd, J=6.0 Hz and 1.2 Hz, Py2H, 2H), 8.96 (d, J=8.0 Hz, Ar1H, 1H), 9.11 (s, ArH, 1H)

Step 7:

The carboxylic acid ERS-7 (0.32 g, 0.817 mmol) was placed in a 50 mL recovery flask, and dissolved by adding N,N-dimethylacetamide (3.2 mL, 10 V). Potassium cyanide (58 mg, 0.898 mmol) was added, and the mixture was heated and stirred in an oil bath at a temperature of 160° C. for 2 hours. The reaction liquid was cooled to room temperature, and ethyl acetate (20 mL), tetrahydrofuran (10 mL), and water (10 mL) were added to perform liquid separation. The separated organic layer was washed with water, washed with saturated saline, and then dried over anhydrous magnesium sulfate. After separation by filtration, the solvent was evaporated under reduced pressure. Diisopropyl ether (5 mL) was added to the crude product, and the solid was taken by filtration, washed with diisopropyl ether (2 mL), and then dried under reduced pressure at 50° C. for 3 hours. The resultant crude product was dissolved in ethanol (2.7 mL) under heating at 80° C., and water (2.7 mL) was added dropwise to the solution. As the mixture was left to cool to room temperature, a crystal precipitated. Water (2.7 mL) was slowly added, and the mixture was further stirred for 1.5 hours. The solid was taken by filtration, washed with ethanol:water (1:2) (1 mL), and then dried under reduced pressure at 50° C. for 3 hours. 0.110 g of a decarboxylation product ERS-8 (1-(2-chloro-5-methylphenyl)-4-oxo-7-(4-pyridyl)-naphthyridine) was obtained as pale yellow powder in a yield of 38.7%.

Data: ¹HNMR (DMSO-d6, δ in ppm): 2.08 (s, ArCH3, 3H), 6.36 (d, J=8.0 Hz, ArH, 1H), 7.55-7.67 (m, Ar3H, 3H), 7.76 (dd, J=4.4 Hz and 1.2 Hz, Py2H, 2H), 8.15 (d, j=8.0 Hz, ArH, 1H), 8.22 (d, J=8.4 Hz, ArH, 1H), 8.67 (dd, J=4.4 Hz and 1.2 Hz, Py2H, 2H), 8.71 (d, J=8.0 Hz, Ar1H, 1H), LC-MS Calcd. for C20H14ClN3O: 347, Found: 348 [M+H].

### (Example 2) Synthesis of Compound (No. 1) of the Present Invention

### (1) Synthesis of 2,6-dichloro-3-(β-(N,N-dimethylamino)acryloyl-pyridine (8)

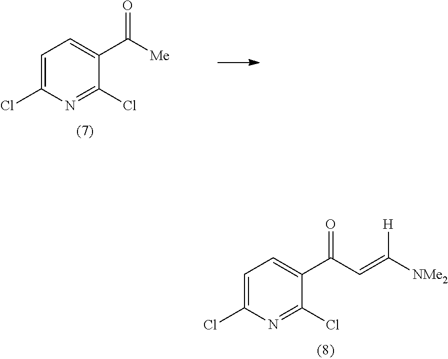 [SMILES: CC(=O)c1ccc(Cl)nc1Cl ; CN(C)/C=C/C(=O)c1ccc(Cl)nc1Cl]

1.9 g (10 mM) of 3-acetyl-2,6-dichloropyridine (7) is weighed and dissolved in 20 mL of dioxane. Under an acidic condition with hydrochloric acid, 1.59 g (12 mM) of N,N-dimethylformamide dimethyl acetal (DMFDA) is added, and reaction treatment is performed according to (1) of Example 3 to be described later to produce 2,6-dichloro-3-(β-(N,N-dimethylamino)acryloyl-pyridine (8).

### (2) Synthesis of 2,6-dichloro-3-(β-(N-2-chloro-5-methyl-phenylamino)acryloyl-pyridine (9)

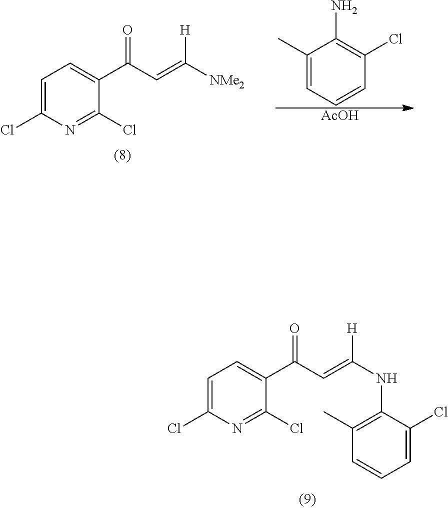

2.45 g (10 mM) of 2,6-dichloro-3-(β-(N,N-dimethylamino)acryloyl-pyridine (8) is weighed and dissolved in 20 mL of DMF, then, 2.83 g (20 mM) of 2-chloro-5-methyl-phenylamine is added, and reaction treatment is performed according to (2) of Example 3 to be described later to produce 2,6-dichloro-3-(β-(N-2-chloro methyl-phenylamino)acryloyl-pyridine (9).

### (3) Synthesis of 1-(2-chloro-5-methylphenyl)-4-oxo-7-chloro-naphthyridine (10)

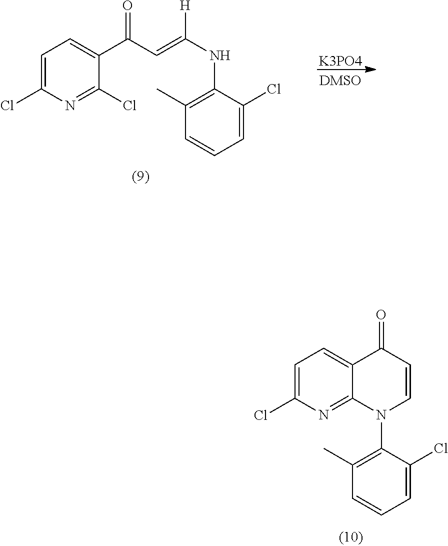 [SMILES: Cc1cccc(Cl)c1N/C=C/C(=O)c1ccc(Cl)nc1Cl ; "K3PO4" ; Cc1cccc(Cl)c1-n1ccc(=O)c2ccc(Cl)nc21]

1.70 g (5 mM) of 2,6-dichloro-3-(β-(N-2-chloro-5-methyl-phenylamino)acryloyl-pyridine (9) is dissolved in 25 mL of DMSO, then, 2.12 g (10 mM) of potassium phosphate (K₃PO₄) is added, and the mixture is heated and stirred at 100° C. for 30 minutes. Reaction treatment is performed according to (3) of Example 3 to be described later and the known literature (K. Awasaguchi et al, Synlett, 2012, 3, 448) to produce a crystal of 1-(2-chloro-5-methylphenyl)-4-oxo-7-chloro-naphthyridine (10).

### (4) Synthesis of 1-(2-chloro-5-methylphenyl)-4-oxo-7-(4-pyridyl)-naphthyridine (11) (Compound No. 1 of the present invention)

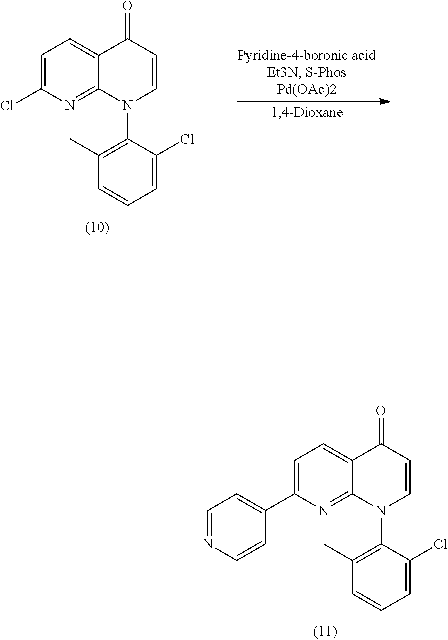 [SMILES: Cc1cccc(Cl)c1-n1ccc(=O)c2ccc(Cl)nc21 ; Cc1cccc(Cl)c1-n1ccc(=O)c2ccc(-c3ccncc3)nc21]

1.74 g (5 mM) of 1-(2-chloro-5-methylphenyl)-4-oxo-7-chloro-naphthyridine (10) is weighed and dissolved in 50 mL of dioxane, 1.40 g (10 mM) of pyridine-4-boronic acid, 2.25 g (10 mM) of palladium acetate, 4.11 g (10 mM) of 2-dicyclohexylphosphino-2′,6′-dimethoxybiphenyl (S-Phos), and 0.59 g (10 mM) of triethylamine are added, and the mixture is heated to reflux for 5 hours. After cooling, the reaction liquid is diluted with ethyl acetate, and washed with saturated saline. The ethyl acetate solution is dried over magnesium sulfate, and the solvent is evaporated to give a residue. The residue is purified by column chromatography to produce a crystal of 1-(2-chloro-5-methylphenyl)-4-oxo-7-(4-pyridyl)-naphthyridine (11) (Compound No. 1 of the present invention).

### (Example 3) Synthesis of Compound (No. 4) of the Present Invention

### (1) Synthesis of β-(N,N-dimethylamino)acryloyl-4-bromo-2-fluorobenzene (3)

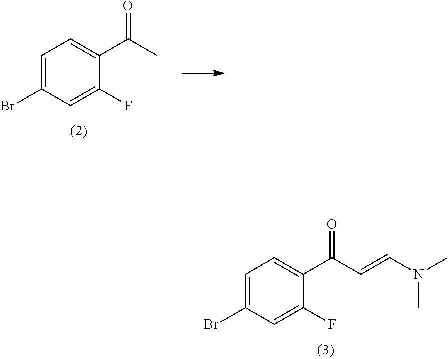

2.17 g (10 mM) of 4-bromo-2-fluoroacetophenone (2) was weighed and dissolved in 20 mL of dioxane. Under an acidic condition with hydrochloric acid, 1.46 g (11 mM) of N,N-dimethylformamide dimethyl acetal (DMFDA) was added, and the mixture was heated to reflux for 8 hours. The reaction solution is poured into an aqueous solution of sodium hydrogen carbonate, and extracted with ethyl acetate. The extract was washed with saturated saline, and dried over magnesium sulfate. The solvent is evaporated under reduced pressure to produce β-(N,N-dimethylamino)acryloyl-4-bromo-2-fluorobenzene (3) in an oily form. The synthesis may be performed according to the known literature (Radl, S. et al, Collect. Czech. Chem. Commun., 2004, 69, 822).

### (2) Synthesis of β-(phenyl-1,1-dimethylmethylamino)acryloyl bromo-2-fluorobenzene (4)

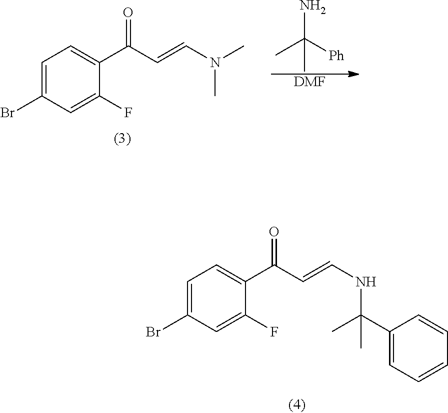

2.70 g (10 mM) of β-(N,N-dimethylamino)acryloyl-4-bromo-2-fluorobenzene (3) is weighed and dissolved in 20 mL of DMF, 2.50 g (20 mM) of phenyl-1,1-dimethylmethylamine is added, and the mixture is heated in the presence of acetic acid at 50° C. for 2 hours. The reaction solution is poured into an aqueous solution of sodium hydrogen carbonate, and extracted with ethyl acetate. The extract was washed with saturated saline, and dried over magnesium sulfate. The solvent is evaporated under reduced pressure to produce 13-(phenyl-1,1-dimethylmethylamino)acryloyl-4-bromo-2-fluorobenzene (4).

### (3) Synthesis of 1-(phenyl-1,1-dimethylmethyl)-4-oxo-7-bromoquinoline (5)

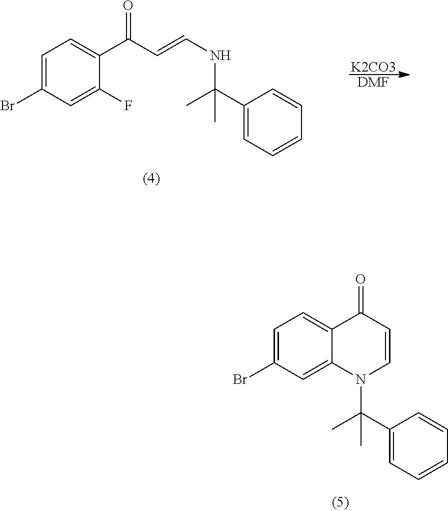

1.75 g (5 mM) of β-(phenyl-1,1-dimethylmethylamino)acryloyl-4-bromo-2-fluorobenzene (4) is dissolved in 50 mL of DMF, then, 1.38 g (10 mM) of potassium carbonate (K₂CO₃) is added, and the mixture is heated and stirred at 80° C. for 30 minutes. The reaction solution was poured into water, and extracted with ethyl acetate. The extract is washed with saturated saline, and dried over magnesium sulfate. The solvent is evaporated under reduced pressure, and purified by column chromatography to produce a crystal of 1-(phenyl-1,1-dimethylmethyl)-4-oxo-7-bromoquinoline (5). The synthesis may be performed according to the known literature (K. Awasaguchi et al, Synlett, 2012, 3, 448).

### (4) Synthesis of 1-(phenyl-1,1-dimethylmethyl)-4-oxo-7-(4-pyrazolyl)-quinoline (6)

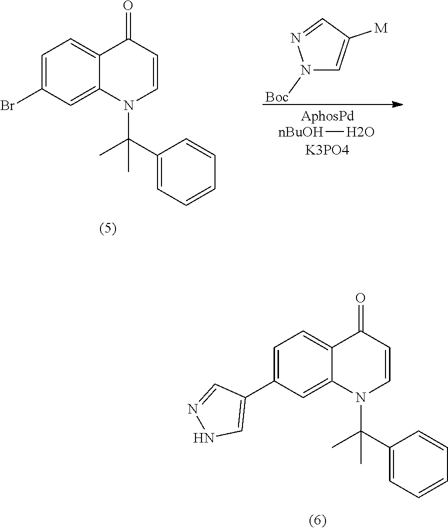

A 4-pyrazolyl group is introduced into the bromo group at the 7-position of 1-(phenyl-1,1-dimethylmethyl)-4-oxo-7-bromoquinoline (5) through use of a Suzuki-Miyaura coupling reaction. 1.71 g (5 mM) of the (5) is weighed and dissolved in 50 mL of dioxane, then, 2.12 g (10 mM) of 2-Boc-pyrazole-4-boronic acid, 2.25 g (10 mM) of palladium acetate, 4.11 g (10 mM) of 2-dicyclohexylphosphino-2′,6′-dimethoxybiphenyl (S-Phos), 2.12 g (10 mM) of tripotassium phosphate, and n-butanol-water are added, and the mixture is heated to reflux. After cooling, the reaction liquid is diluted with ethyl acetate, and washed with saturated saline. The ethyl acetate solution is dried over magnesium sulfate, and the solvent is evaporated to give a residue. Purification is performed by column chromatography to produce a crystal of 1-(phenyldimethylmethyl)-4-oxo-7-(4-pyrazolyl)-quinoline (6) (Compound No. 4 of the present invention).

Compounds No. 2, 3, and 5 to 7 shown below of the present invention may be similarly synthesized.

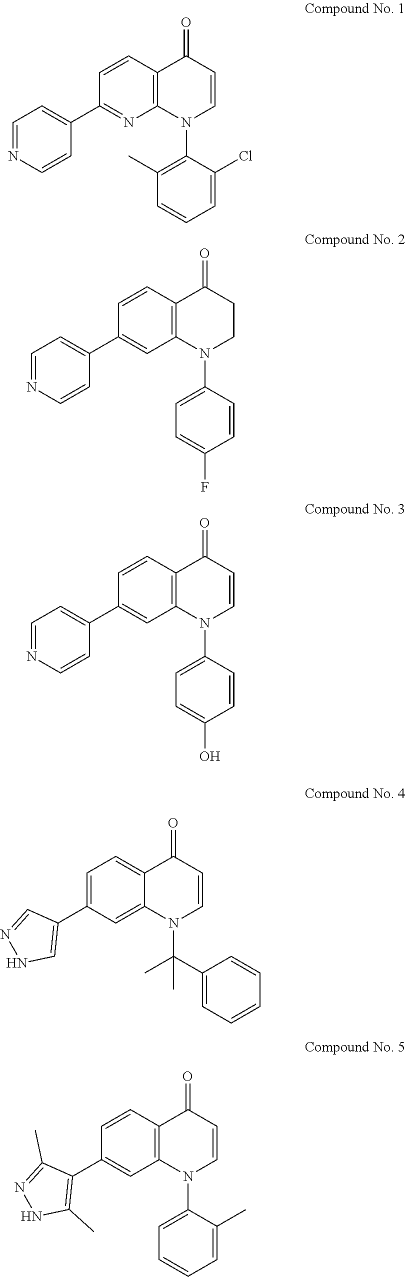

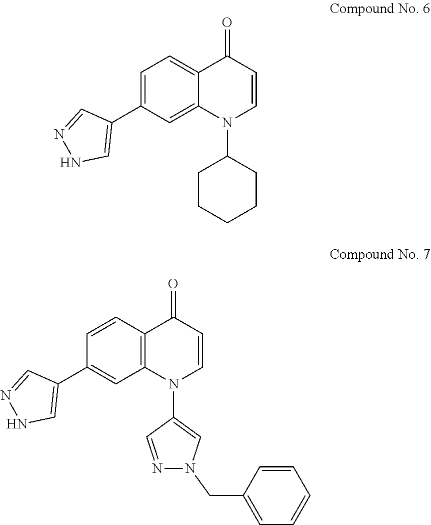 [SMILES: O=c1ccn(C2CCCCC2)c2cc(-c3cn[nH]c3)ccc12 ; O=c1ccn(-c2cnn(Cc3ccccc3)c2)c2cc(-c3cn[nH]c3)ccc12]

(Test Example 1) Evaluation of eIF2α phosphorylation-promoting activity of compound of the present invention

### (1) Materials and Reagents:

Host cells: 293A cells
Evaluation compound: Compound No. 1 of the present invention was used, and 137 μg thereof was dissolved in 20 μL of DMSO to prepare a 20 mM solution.
Thapsigargin: (Cayman Chemical Company)

10 mg was dissolved in 76.8 mL of DMSO to prepare a 200 μM solution.
Halofuginon: (Cayman Chemical Company)

5 mg was dissolved in 554.2 μL of DMSO to prepare a 20 mM solution.

### (2) Method:

The above-mentioned 293A cells were seeded in a 96-well plate at 5×10⁵ cells/well, and were cultured overnight in a DMEM solution of 10% fetal bovine serum (FBS). The next day, the medium was changed to a DMEM solution of 10% fetal bovine serum (FBS) containing Compound No. 1 at 5 μM, halofuginone at 50 nM, or thapsigargin at 200 nM, and the cells were cultured at 37° C. for 16 hours. After the cells had been washed with a PBS buffer, the cells were lysed with a RIPA buffer to extract proteins, and the proteins were separated according to size by an electrophoresis method of SDS-PAGE. The expression amount of an endoplasmic reticulum stress marker was measured by a western blotting method.

### (3) Results:

The expression amounts of eIF2α and β-actin as well as phosphorylated eIF2α serving as the endoplasmic reticulum stress marker were determined. The results are shown in FIG. 3. It was shown that Compound No. 1 of the present invention had an eIF2α phosphorylation-promoting effect like thapsigargin and halofuginone.

(Test Example 2) Evaluation of Topoisomerase-inhibiting Activity of Compound of the Present Invention

### (1) Materials and Reagents:

Evaluation compound: Compound No. 1 of the present invention was used, and 137 μg thereof was dissolved in 20 μL of DMSO to prepare a 20 mM solution.
Camptothecin: (FUJIFILM Wako Pure Chemical Corporation)

100 mg was dissolved in 57.4 mL of DMSO to prepare a 5 mM solution.
Etoposide: (FUJIFILM Wako Pure Chemical Corporation)

25 mg was dissolved in 424.8 μL of DMSO to prepare a 100 mM solution.
Human topoisomerase I/DNA relaxation assay kit HRA100KE (Manufactured by ProFoldin)
Human topoisomerase II/DNA relaxation assay kit HDC020K (Manufactured by ProFoldin)

### (2) Method:

Measurement was performed in accordance with an instruction manual of ProFoldin. First, 5 μL of an evaluation sample solution (100 μM), 5 μL of a ligation DNA solution (30 μg/mL), 5 μL of an ATP solution (2 mM), and 0.5 μL of a human topoisomerase (1,000 U/mL) were added to 34.5 μL of distilled water in a reaction tube, and the mixture was subjected to a reaction at room temperature for from 30 minutes to 60 minutes. 5 μL of 0.5 M EDTA was added to stop the reaction. After the reaction, centrifugation was performed at 1,300 rpm for 30 seconds, and the reaction liquid was poured into a spin-column. The spin-column was rotated at 1,300 rpm for 2 minutes, and the liquid that had flowed out of the column was poured into a 96-well black plate. The liquid that had flowed out was subjected to a reaction by adding a fluorescent dye solution (up to 150 μL). The intensity of fluorescence excited with light at 485 nm was measured at 535 nm.

### (3) Results:

As shown in FIG. 4, it was revealed that Compound No. 1 of the present invention did not react with human topoisomerase I or human topoisomerase II, and did not show topoisomerase-inhibiting activity. As a result, it was revealed that Compound No. 1 of the present invention, though being a compound having a quinolone skeleton, was free of antibacterial activity or anticancer activity based on topoisomerase-inhibiting activity.

Meanwhile, in this test system, camptothecin remarkably reduced topoisomerase I activity, and etoposide remarkably reduced topoisomerase II activity.

(Test Example 3) Evaluation of Th17 cell differentiation-suppressing activity of compound of the present invention

### (1) Materials and Reagents:

Evaluation compound: Compound No. 1 of the present invention was used, and 137 μg thereof was dissolved in 20 μL of DMSO to prepare a 20 mM solution.
Halofuginon: (Cayman Chemical Company)

5 mg was dissolved in 554.2 μL of DMSO to prepare a 20 mM solution.
Th17 cell inducer: A Th17 cell differentiation induction kit containing IL-6 and TGF131 as main components (CellXVivo Mouse Th17 Cell Differentiation Kit: R&D Systems)
Mouse helper T cell differentiation induction kit CDK017 (Techne Corporation)

### (2) Method:

Measurement was performed in accordance with an instruction manual of Techne Corporation. First, a cell culture plate was coated with an anti-hamster CD3 antibody. Mouse spleen cells were isolated into single cells, and mouse naive CD4⁺ T cells were isolated using a mouse naive CD4⁺ T cell isolation kit (MagCellect). The number of cells was counted. The isolated mouse naive CD4⁺ T cells were dispersed in a mouse Th17 differentiation medium at 1×10⁶ cells/mL. Compound No. 1 and halofuginone were added to the mouse Th17 differentiation medium at 5 μM and 50 nM, respectively. Next, the medium having the cells dispersed therein was added to the plate coated with the anti-hamster CD3 antibody, and humidified culture was continued for 3 days at 37° C. in the presence of 5% CO₂. On the 3rd day, the medium was refreshed by adding an equal amount of the mouse Th17 differentiation medium without removing the original culture medium. Humidified culture was continued for 2 days at 37° C. in the presence of 5% CO₂. After the 5 days of differentiation culture, the differentiated mouse Th17 cells were subjected to flow cytometry to analyze the expression of cytokines, and the number of cells differentiated into Th17 cells was determined.

### (3) Results:

As shown in FIG. 5, Compound No. 1 of the present invention remarkably suppressed differentiation from mouse naive CD4⁺ T cells into Th17 cells to a degree comparable to that of halofuginone being a known compound capable of inhibiting the induction of differentiation into Th17 cells.

Thus, it was revealed that the compound of the present invention was effective for an autoimmune disease or a persistent inflammatory disease like halofuginone.

### INDUSTRIAL APPLICABILITY

The quinolone skeleton compound of the present invention acts as an eIF2α phosphorylation promotor. Further, the quinolone skeleton compound of the present invention is free of topoisomerase-inhibiting activity serving as a basis for antibacterial activity, and further has Th17 cell differentiation-suppressing activity. Accordingly, the compound of the present invention serves as a drug for preventing or ameliorating endoplasmic reticulum stress, the drug being a novel mother nucleus compound that has not been known heretofore, and is effective as a therapeutic agent for an autoimmune disease or a persistent inflammatory disease. Further, the compound of the present invention is also useful as a preventive or therapeutic agent for metabolic disease, cranial nerve disease, cancer, and immunological disease in each of which endoplasmic reticulum stress is involved.

1. A compound represented by the general formula (1) or a pharmacologically acceptable salt thereof: 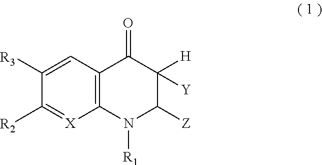
where: R₁ represents a substituted or unsubstituted aryl group, a substituted or unsubstituted aralkyl group, a substituted or unsubstituted cycloalkyl group, or a substituted or unsubstituted aromatic heterocyclic group; R₂ represents a substituted or unsubstituted aromatic heterocyclic group; R₃ represents a hydrogen atom, a hydroxy group, or a halogen atom; substituents of the aryl group, the aralkyl group, and the cycloalkyl group of R₁ are each independently a halogen atom, a hydroxy group, a lower alkyl group having 1 to 4 carbon atoms, or an aralkyl group; substituents of the aromatic heterocyclic groups of R₁ and R₂ are each independently a substituted or unsubstituted benzyl group, a hydroxy group, an alkyl group having 1 to 4 carbon atoms, a halogen atom, or an alkyloxy group having 1 to 4 carbon atoms; X represents a nitrogen atom or a methine group; and Y and Z both represent a hydrogen atom or are paired to represent a single bond.

2. The compound or the pharmacologically acceptable salt thereof according to claim 1, wherein the substituted or unsubstituted aryl group is a 2-methylphenyl group, a p-hydroxyphenyl group, a p-fluorophenyl group, or a 2-chloro-5-methylphenyl group.

3. The compound or the pharmacologically acceptable salt thereof according to claim 1, wherein the substituted or unsubstituted aralkyl group is a benzyl group or a phenyl-1,1-dimethylmethyl group.

4. The compound or the pharmacologically acceptable salt thereof according to claim 1, wherein the substituted or unsubstituted cycloalkyl group is a cyclohexyl group.

5. The compound or the pharmacologically acceptable salt thereof according to claim 1, wherein the substituted or unsubstituted aromatic heterocyclic groups are each a 4-pyridyl group, a 4-pyrazolyl group, a 3,5-dimethyl-4-pyrazolyl group, or a 1-N-benzyl-4-pyrazolyl group.

6. The compound or the pharmacologically acceptable salt thereof according to claim 1, wherein the X represents a nitrogen atom.

7. The compound or the pharmacologically acceptable salt thereof according to claim 1, wherein the X represents a methine group.

8. The compound or the pharmacologically acceptable salt thereof according to claim 1, wherein the Y and Z are paired to represent a single bond.

9. The compound or the pharmacologically acceptable salt thereof according to claim 1, wherein R₁ represents a substituted or unsubstituted aryl group, R₂ represents an unsubstituted aromatic heterocyclic group, R₃ represents a hydrogen atom, X represents a nitrogen atom, and Y and Z are paired to represent a single bond.

10. The compound or the pharmacologically acceptable salt thereof according to claim 1, wherein R₁ represents a substituted or unsubstituted phenyl group, R₂ represents an unsubstituted pyridyl group, R₃ represents a hydrogen atom, X represents a nitrogen atom, and Y and Z are paired to represent a single bond.

11. The compound or the pharmacologically acceptable salt thereof according to claim 1, wherein R₁ represents a phenyl group having a substituent, R₂ represents an unsubstituted pyridyl group, R₃ represents a hydrogen atom, X represents a nitrogen atom, and Y and Z are paired to represent a single bond.

12. The compound or the pharmacologically acceptable salt thereof according to claim 1, wherein R₁ represents a phenyl group having as substituents an alkyl group having 1 to 4 carbon atoms and a halogen atom, R₂ represents an unsubstituted pyridyl group, R₃ represents a hydrogen atom, X represents a nitrogen atom, and Y and Z are paired to represent a single bond.

13. A method of promoting eIF2α phosphorylation comprising administering the compound or the pharmacologically acceptable salt thereof of claim 1 in an amount effective for promoting eIF2α phosphorylation to a human having a disrupted or reduced endoplasmic reticulum stress response or integrated stress response.

14. The method according to claim 13, wherein the compound or the pharmacologically acceptable salt thereof is administered in an amount effective for promoting eIF2α phosphorylation and not effective for inhibiting topoisomerase.

15. The method according to claim 13, wherein the compound or the pharmacologically acceptable salt thereof is administered in an amount effective for suppressing Th17 cell differentiation along with promoting eIF2α phosphorylation.

16. A method of preventing or treating a disease in a subject, said method comprising administering the compound or the pharmacologically acceptable salt thereof of claim 1 in an amount effective for preventing or treating the disease to a patient with the disease, wherein the disease is selected from the group consisting of an autoimmune disease, a persistent inflammatory disease, a neurodegenerative disease, cancer, diabetes, and arteriosclerosis.
